(19) United States
(12) Patent Application Publication
(10) Pub. No.: US 2010/0216857 A1
(43) Pub. Date: Aug. 26, 2010

(54) METHOD OF TREATING MOTOR DISORDERS WITH 4-(1-(2,3-DIMETHYLPHENYL)ETHYL)-1H-IMIDAZOLE-2(3H)-THIONE

(76) Inventors: Lauren M.B. Luhrs, Rancho Santa Margarita, CA (US); Daniel W. Gil, Corona Del Mar, CA (US); John E. Donello, Dana Point, CA (US)

Correspondence Address: ALLERGAN, INC., 2525 DUPONT DRIVE, T2-7H, IRVINE, CA, 92612-1599, US

(21) Appl. No.: 12/680,656

(22) PCT Filed: Oct. 14, 2008
(86) PCT No.: PCT/US08/79771

§ 371 Date: Mar. 29, 2010

Related U.S. Application Data
(60) Provisional application No. 60/981,010, filed on Oct. 18, 2007.

(51) Int. Cl. A61K 31/4164; A61P 25/16; A61P 25/00

(52) U.S. Cl. 514/392

(57) ABSTRACT

Disclosed herein is a method of treating motor disorders comprising administering to a subject in need of such treatment 4-(1-(2,3-dimethylphenyl)ethyl)-1H-imidazole-2(3H)-thione.

### CROSS-REFERENCE

This application claims the benefit of U.S. application Ser. No. 60/981,010, filed Oct. 18, 2007, which is hereby incorporated by reference in its entirety.

Disclosed herein is a method of treating motor disorders by administering to a subject 4-(1-(2,3-dimethylphenyl)ethyl)-1H-imidazole-2(3H)-thione.

### DETAILED DESCRIPTION OF THE INVENTION

### 4-(1-(2,3-dimethylphenyl)ethyl)-1H-imidazole-2(3H)-thione

The compound of the invention, 4-(1-(2,3-dimethylphenyl)ethyl)-1H-imidazole-2(3H)-thione, is described in U.S. Pat. No. 7,141,597, the contents of which are incorporated by reference herein. The compound is an alpha-2 adrenergic receptor agonist having the following structure:

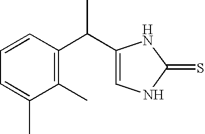

It occurs as two enantiomers. The R-enantiomer is depicted here on the left; the S-enantiomer on the right:

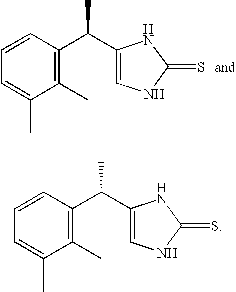

One can use in the methods of the invention either enantiomer, or a mixture of both, as well as any pharmaceutically acceptable salt or prodrug of these compounds.

### Pharmaceutically Acceptable Salts

The compound of the invention may be used as its pharmaceutically acceptable salt.

A “pharmaceutically acceptable salt” is any salt that retains the activity of the parent compound and does not impart any additional deleterious or untoward effects on the subject to which it is administered and in the context in which it is administered compared to the parent compound. A pharmaceutically acceptable salt also refers to any salt which may form in vivo as a result of administration of an acid, another salt, or a prodrug which is converted into an acid or salt.

Pharmaceutically acceptable salts of acidic functional groups may be derived from organic or inorganic bases. The salt may comprise a mono or polyvalent ion. Of particular interest are the inorganic ions lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

### Prodrugs

One can use in the methods of the invention a prodrug of the compound of 4-(1-(2,3-dimethylphenyl)ethyl)-1H-imidazole-2(3H)-thione.

A “prodrug” is a compound which is converted to a therapeutically active compound after administration, and the term should be interpreted as broadly herein as is generally understood in the art. While not intending to limit the scope of the invention, conversion may occur by hydrolysis of an ester group or some other biologically labile group. Generally, but not necessarily, a prodrug is inactive or less active than the therapeutically active compound to which it is converted. Ester prodrugs of the compounds disclosed herein are specifically contemplated. An ester may be derived from a carboxylic acid of C1 (i.e., the terminal carboxylic acid of a natural prostaglandin), or an ester may be derived from a carboxylic acid functional group on another part of the molecule, such as on a phenyl ring. While not intending to be limiting, an ester may be an alkyl ester, an aryl ester, or a heteroaryl ester. The term alkyl has the meaning generally understood by those skilled in the art and refers to linear, branched, or cyclic alkyl moieties. C₁₋₆ alkyl esters are particularly useful, where alkyl part of the ester has from 1 to 6 carbon atoms and includes, but is not limited to, methyl, ethyl, propyl, isopropyl, n-butyl, sec-butyl, iso-butyl, t-butyl, pentyl isomers, hexyl isomers, cyclopropyl, cyclobutyl, cyclopentyl, cyclohexyl, and combinations thereof having from 1-6 carbon atoms, etc.

4-(1-(2,3-dimethylphenyl)ethyl)-1H-imidazole-2(3H)-thione may be either synthetically produced, or may be produced within the body after administration of a prodrug. Hence, “4-(1-(2,3-dimethylphenyl)ethyl)-1H-imidazole-2(3H)-thione” encompasses both compounds produced by a manufacturing process and those compounds formed in vivo only when another drug administered.

### Dose

The precise dose and frequency of administration depends on the severity and nature of the patient's condition, on the manner of administration, on the potency and pharmacodynamics of the particular compound employed, and on the judgment of the prescribing physician. Determining dose is a routine matter that is well within the capability of someone of ordinary skill in the art. In general, the compound of the invention is administered in therapeutically effective doses, that is, at a dose that is sufficient to produce the desired therapeutic effect.

### Excipients and Dosage Forms

Those skilled in the art will readily understand that the compound of the invention can be admixed with pharmaceutically acceptable excipients which are well known in the art.

A pharmaceutical composition to be administered systemically may be confected as a powder, pill, tablet or the like, or as a solution, emulsion, suspension, aerosol, syrup or elixir suitable for oral or parenteral administration or inhalation.

For solid dosage forms or medicaments, non-toxic solid carriers include, but are not limited to, pharmaceutical grades of mannitol, lactose, starch, magnesium stearate, sodium saccharin, the polyalkylene glycols, talcum, cellulose, glucose, sucrose and magnesium carbonate. The solid dosage forms may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in U.S. Pat. No. 4,256,108, U.S. Pat. No. 4,166,452, and U.S. Pat. No. 4,265,874 to form osmotic therapeutic tablets for control release. Liquid pharmaceutically administrable dosage forms can, for example, comprise a solution or suspension of one or more of the presently useful compounds and optional pharmaceutical adjutants in a carrier, such as for example, water, saline, aqueous dextrose, glycerol, ethanol and the like, to thereby form a solution or suspension. If desired, the pharmaceutical composition to be administered may also contain minor amounts of nontoxic auxiliary substances such as wetting or emulsifying agents, pH buffering agents and the like. Typical examples of such auxiliary agents are sodium acetate, sorbitan monolaurate, triethanolamine, sodium acetate, triethanolamine oleate, etc. Actual methods of preparing such dosage forms are known, or will be apparent, to those skilled in this art; for example, see Remington's Pharmaceutical Sciences, Mack Publishing Company, Easton, Pa., 16th Edition, 1980. The composition of the formulation to be administered, in any event, contains a quantity of one or more of the presently useful compounds in an amount effective to provide the desired therapeutic effect.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol and the like. In addition, if desired, the injectable pharmaceutical compositions to be administered may also contain minor amounts of non-toxic auxiliary substances such as wetting or emulsifying agents, pH buffering agents and the like.

### Motor Disorders

4-(1-(2,3-dimethylphenyl)ethyl)-1H-imidazole-2(3H)-thione is useful in motor disorders. To “treat,” as used here, means to deal with medically. It includes administering the compound of the invention to prevent the onset of a condition, to diminish its severity, and to prevent its reoccurrence. The inventors have discovered that the compound of the invention may be used to treat motor disorders without causing the sedation that ordinarily accompanies the administration of alpha-2 agonists.

A “motor disorder,” as that term is used here, is any condition in which a subject experiences involuntary, undesirable movements that are independent of any deficits in sensorimotor gating; that is, the movement is not the result of abnormal motor output in response to sensory input information.

In one embodiment, the motor disorder is mediated by changes (for example, an increase or a decrease) in the availability or utilization of dopamine in the nervous system; hence, compounds of the invention may be used to treat motor disorders associated with hyper- or hypo-dopamine conditions of the nervous system.

Exemplary motor disorders which may be treated with the compounds of the invention include, for example, L-Dopa-induced dyskinesias, tardive dyskinesias, cervical dystonia, spinal torticollis, blepharospasm/Meige's disease, restless leg syndrome, essential tremor, rigidity (Parkinson's disease-associated or otherwise specified), ataxic disorder, spasticity.

### EXAMPLES

The invention is illustrated by the following examples. This is provided for illustration only; many more embodiments are possible.

4-(1-(2,3-dimethylphenyl)ethyl)-1H-imidazole-2(3H)-thione was evaluated in tests of amphetamine-induced hyperlocomotion, cocaine-induced hyperlocomotion, and nigrostriatal-lesion-induced rotational behavior following amphetamine administration.

### General Findings

Amphetamine-induced and cocaine-induced hyperactivity are models of increased dopamine-mediated locomotion. The nigrostriatal lesion-induced rotation model exploits the imbalance in dopaminergic innervation following a unilateral lesion of the dopamine-containing substantia nigra pars compacta (SNc), thereby creating a hemiparkinsonian condition. The subsequent rotational behavior in these animals induced by various drugs can be used to infer the level of dopamine imbalance on the lesioned and unlesioned side of the brain. In amphetamine- and cocaine-induced hyperactivity, the compound of the invention was able to effectively inhibit increased locomotion associated with psychostimulant administration. In addition, this compounds was also able to inhibit amphetamine-induced rotational behavior in nigrostriatal-lesioned animals.

Importantly, the compounds is orally active, and therefore could be administered in solution, tablet or capsule.

### Amphetamine-induced Hyperlocomotion

Table 1, below, indicates the locomotor responses of mice treated with various combinations of 4-(1-(2,3-dimethylphenyl)ethyl)-1H-imidazole-2(3H)-thione and the psychostimulant amphetamine. Drug 1 was administered 30 minutes after the animal was placed in the open field. Drug 2 was administered subcutaneously (SC) at 45 minutes after the animal was placed in the open field (15 minutes after drug 1). AUC indicates the total amount of locomotor activity after administration of drug 2. “% decrease activity” indicates the percentage decrease in total activity relative to the Vehicle+Amphetamine group. ⋄ significant difference (P<0.05) relative to Vehicle+ Amphetamine treated animals. * indicates significant difference (P ≦0.05) relative to Vehicle+Vehicle treated animals.

| | | | | TOTAL ACTIVITY | % DECREASE |
|---|---|---|---|---|---|
| | | | | POST- | ACTIVITY REL |
| | DOSE | | DRUG 2 | AMPHETAMINE | TO |
| DRUG 1 | (MG/KG) | ROUTE | (SC) | (AUC) | VEH + AMPH |
| Vehicle | 0 | IP | Vehicle | 3328.83 ± 824 | 73%^{⋄} |
| Vehicle | 0 | IP | Amph | 13341 ± 1607.2 | n/a |
| Haloperidol | 0.2 | IP | Amph | 1123.16 ± 280.11 | 91%^{⋄}* |
| Compound | 0.01 | PO | Amph | 9093.3 ± 983.8 | 32%* |
| of the | | | | | |
| invention | | | | | |
| Compound | 0.03 | PO | Amph | 3783.3 ± 214.2 | 72%^{⋄} |
| of the | | | | | |
| invention | | | | | |
| Compound | 0.1 | PO | Amph | 5036.0 ± 654.2 | 63%^{⋄} |
| of the | | | | | |
| invention | | | | | |
| Compound | 0.1 | PO | Amph | 3759.1 ± 685.4 | 72%^{⋄} |
| of the | | | | | |
| invention | | | | | |

### Cocaine-induced Hyperlocomotion

Table 2, below, indicates the locomotor responses of mice treated with various combinations of 4-(1-(2,3-dimethylphenyl)ethyl)-1H-imidazole-2(3H)-thione and the psychostimulant cocaine. Drug 1 was administered 30 minutes after the animal was placed in the open field. Drug 2 was administered subcutaneously (SC) at 45 minutes after the animal was placed in the open field (15 minutes after drug 1). AUC indicates the total amount of locomotor activity after administration of drug 2. “% decrease activity” indicates the percentage decrease in total activity relative to the Vehicle+Cocaine group. ⋄ indicates significant difference (P≦0.05) relative to Vehicle+Cocaine treated animals. * indicates significant difference (P≦0.05) relative to Vehicle+Vehicle treated animals.

| | | | | TOTAL ACTIVITY | |
|---|---|---|---|---|---|
| | DOSE | | DRUG 2 | POST-COCAINE | % DECREASE |
| DRUG 1 | (MG/KG) | ROUTE | (SC) | (AUC) | ACTIVITY |
| Vehicle | 0 | PO | Vehicle | 2861.2 ± 497.33 | 83%^{⋄} |
| Vehicle | 0 | PO | Cocaine | 10391 ± 1208.32 | n/a |
| Compound | 0.01 | PO | Cocaine | 4904 ± 1832.1 | 53%^{⋄} |
| of the | | | | | |
| invention | | | | | |
| Compound | 0.03 | PO | Cocaine | 7260.8 ± 1756.5 | 31%* |
| of the | | | | | |
| invention | | | | | |
| Compound | 0.1 | PO | Cocaine | 3754.3 ± 1281.9 | 64%^{⋄} |
| of the | | | | | |
| invention | | | | | |

### Nigrostriatal-lesion-induced Rotational Behavior Following Amphetamine Administration

Table 3, below, indicates the changes in rotational behavior in nigrostriatal-lesioned rats treated with the compound of the invention and the psychostimulant amphetamine. Drug 1 was administered 15 minutes before amphetamine, and 10 minutes post-amphetamine, the animals were placed into the rotometer for 30 or 60 minutes. “% decrease rotations” indicates the percentage decrease in total rotations relative to the Vehicle+Amphetamine group. ⋄ indicates significant difference (P≦0.05) relative to Vehicle+Amphetamine treated animals. * indicates significant difference (P+0.05) relative to Vehicle+Vehicle treated animals.

| | DOSE | | DRUG 2 | % DECREASE |
|---|---|---|---|---|
| DRUG 1 | (MG/KG) | ROUTE | (SC) | ROTATIONS |
| Vehicle | 0 | PO | Vehicle | 91%^{⋄} |
| Vehicle | 0 | PO | Amph | n/a |
| Compound of | 0.03 | PO | Amph | 68%^{⋄} |
| the invention | | | | |
| Compound of | 0.1 | PO | Amph | 68%^{⋄} |
| the invention | | | | |
| Compound of | 1 | PO | Amph | 46%^{⋄}* |
| the invention | | | | |

### Materials and Methods

Amphetamine- or cocaine-induced hyperactivity. Mice were placed in an open field apparatus (FlexField, San Diego Instruments, San Diego, Calif.). After 30 minutes of habituation, they received a vehicle or haloperidol injection or injection of AGN compound followed by an injection of amphetamine (2 mg/kg, s.c.) or cocaine (10 mg/kg, i.p.) at minute 45. Their activity levels were subsequently measured for another 1 hour post-injection. Total activity over each 5 minute bin (21 total bins) was added to establish activity curve over the 105 minute testing.

Amphetamine-induced rotational behavior. Pre-Apomorphine-screened (rotated) rats (with lesions ≧95% DA cell loss) were purchased from Charles Rivers Laboratories. Animals were given ˜4 days to acclimate prior to testing. All animals weighted 250-300 g at the beginning of the study. Animals were treated with various combinations of vehicle or the compound of the invention and amphetamine.

Vehicle or the compound of the invention was administered (p.o.) 15 minutes before amphetamine (2 mg/kg s.c.). Rotational behavior was assessed 15 minutes post-amphetamine administration and was monitored for 60 minutes using a Rotometer system from San Diego Instruments (San Diego, Calif.).

For i.p. administration, the compounds are formulated in H₂O with 0.5% DMSO and given in a volume of 1 ml/kg body weight by injecting into the intraperitoneal cavity. For p.o. administration, the compounds are formulated in H₂O with 0.5% DMSO and given in a volume of 2 ml/kg body weight using a 25-gauge, 1.5 inch gavage needle that is slowly inserted through the esophagus into the stomach.

What is claimed is:

1. A method of treating a motor disorder comprising administering to a subject in need of such treatment a compound having the following structure:

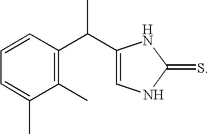

2. The method of claim 1, wherein the method further comprises treating the motor disorder without causing sedation.

3. The method of claim 2, wherein the motor disorder is associated with a hyper- or hypo-dopamine condition of the nervous system.

4. The method of claim 2, wherein the disorder is selected from the group consisting of dystonia, L-Dopa-induced dyskinesias, tardive dyskinesias, cervical dystonia, spinal torticollis, blepharospasm/Meige's disease, restless leg syndrome, essential tremor, rigidity (Parkinson's disease-associated or otherwise specified), ataxic disorder, and spasticity.
